(19) United States
(12) Patent Application Publication
(10) Pub. No.: US 2009/0221227 A1
(43) Pub. Date: Sep. 3, 2009

(54) COMPOST VENTILATION

(75) Inventors: John Jardine, Bury St Edmunds (GB); Simon James Watchorn, Bungay (GB)

Correspondence Address: BOURQUE & ASSOCIATES;INTELLECTUAL PROPERTY ATTORNEYS, P.A., 835 HANOVER STREET, SUITE 301, MANCHESTER, NH, 03104, US

(73) Assignee: Covered System Limited, Ipswich (GB)

(21) Appl. No.: 12/095,411

(22) PCT Filed: Nov. 29, 2006
(86) PCT No.: PCT/GB2006/004454

§ 371 Date: Aug. 25, 2008

(30) Foreign Application Priority Data
Nov. 29, 2005 (GB) 0524255.7

(51) Int. Cl. F24F 7/06; C12M 1/00

(52) U.S. Cl. 454/341; 435/290.1

(57) ABSTRACT

A composting apparatus (1) comprising a compost heap (2) comprising compostable material. There is at least one ventilation pipe (4) and air moving means (6). The ventilation pipe (4) comprising a pipe wall (8) defining a conduit for gas. The ventilation pipe has a connection end (10) and a ventilation portion (12). The ventilation portion has a plurality of aeration holes (14) extending through the pipe wall (8). The ventilation portion (12) is located within the compost heap (2) and the connection end (10) is coupled to the air moving means (6) such that, when active, the air moving means (6) causes movement of air through the aeration holes (14). The invention also extends to a method of ventilation compost.

### BACKGROUND

a. Field of the Invention

The present invention relates to apparatus for composting apparatus and to a method of ventilating a compost heap, in particular to the ventilation of compost heaps and windrows so as to reduce odour.

b. Related Art

Composting of compostable material is typically performed in heaps. These heaps may be located inside buildings or outside. If the compost heap is located outside, it is typically in the form of an elongate row called a windrow. Compost heaps may be agitated or turned during the composting process to aerate the content to ensure efficient composting. Compostable material is used herein to include biodegradable materials such as household waste and other waste materials.

It is known to have vent channels located on the floor of a building in which a compost heap is to be located. The vent channels in the floor either blow air into the base of the compost heap, or draw air from the base of the heap. This air movement through the base of the heap ventilates the heap and therefore promotes more rapid and even composting of the material in the pile and removes the need to manually or mechanically turn or aerate the heap.

However, such vent channels can be costly to build into the, typically concrete, floor. To avoid the cost of building the vents into the floor it is known to locate vent channels on the surface of the floor. However the channels are constructed on the floor they are vulnerable to damage by machinery used to transport the compost material as the compost heap is built over the channels. The channels also tend to fill with debris and liquid leachate from the heap and require frequent cleaning to ensure that the channels are still open for air flow.

It is an object of the present invention to provide improved ventilation of a compost heap.

### SUMMARY OF THE INVENTION

The invention provides a composting apparatus comprising a compost heap comprising compostable material, at least one ventilation pipe and air moving means, the ventilation pipe comprising a pipe wall defining a conduit for gas and the ventilation pipe having a connection end and a ventilation portion, the ventilation portion having a plurality of aeration holes extending through the pipe wall and wherein the ventilation portion is located within the compost heap and the connection end is coupled to the air moving means such that, when active, the air moving means draws air from the, or each, ventilation pipe; and the apparatus further comprising air treatment means for treating the air drawn from the ventilation pipe to reduce odours in the gas before it is released to atmosphere.

Such treatment may involve passing the air through activated charcoal or other filters. The filters preferably reduce, and more preferably, eliminate odours, but may perform other treatments on the air to remove potential toxins or other chemicals.

By having a ventilation pipe with a ventilation portion within the compost heap the ventilation of the heap is more uniform as air is being moved from with the heap rather than at an outer surface of the heap. Aeration of the heap is achieved by drawing air through the ventilation pipe, thereby causing air to be drawn through the compost heap toward the ventilation portion. The drawing of air into the ventilation pipe can also help to prevent the heap from drying out as moisture is drawn into the heap rather than being blown to the external surfaces from which it can easily evaporate. The drawing of air into the heap also help to control odour as the volatile chemicals are retained within the heap rather than being blown outwards.

The aim is to promote aerobic digestion of the material. Such digestion generates heat and this heat tends to concentrate in a top core. By positioning the ventilation portion below the top core and drawing air into the ventilation pipe, the concentration of heat can be drawn downwards towards the ventilation portion by the movement of the air through the ‘hot spot’.

It is preferred that there are a plurality of ventilation pipes having ventilation portions within the compost heap so that the aeration is distributed throughout the compost heap as this helps to avoid ‘hot spots’ or areas of high or low aeration.

The ventilation pipe is preferably arranged substantially vertically within the compost heap such that the connection end extends above an upper surface of the heap. This facilitates connection of the ventilation pipe to the air moving means and can help to prevent the ventilation pipe from filling with compost material which may occur if material can fall through the aeration holes.

The invention also provides a method of ventilating a compost heap using at least one ventilation pipe and an air moving means, the ventilation pipe comprising a pipe wall defining a conduit for gas and the ventilation pipe having a connection end and a ventilation portion, the ventilation portion having a plurality of aeration holes extending through the pipe wall and the method comprising the steps of:
constructing a compost heap comprising compostable materials;
inserting the ventilation portion of the at least one ventilation pipe into the compost heap;
connecting the connection end of the ventilation pipe to the air moving means;
using the air moving means to draw air from the ventilation pipe and draw air through the aeration holes into the ventilation pipe via air treatment means.

It is preferred that the step of inserting the ventilation portion into the compost heap comprises coupling the ventilation pipe to a piler apparatus and using the piler apparatus to force the ventilation pipe into the compost heap. When the ventilation pipe has been forced into the required position, the piler apparatus is released from the ventilation pipe and retrieved from the ventilation pipe. If a plurality of ventilation pipes are being used, this step can be repeated as often as required to insert all the ventilation pipes. The ventilation pipes may be adapted to include a coupling portion adapted to be coupled to a coupling portion of the piler apparatus. The ventilation pipes preferably also include a nose portion adapted to penetrate the compost heap during the piling operation.

It should be understood that the ventilation pipe may be inserted using a variety of methods, for example forcing the ventilation pipes into the heap using a pile driver or similar apparatus.

The ventilation pipes may be inserted into the compost heap as it is constructed, but it is preferred that the ventilation pipes are inserted after construction of the heap as this speeds up the construction of the heap as there is no need to stop construction to arrange the ventilation pipes before the remainder of the heap is constructed.

It is preferred that the piler apparatus is compressed air powered, for example it may be the Grundomat™ from TT UK Limited. Such pilers include a head portion that reciprocates rapidly to drive the piler through the material.

It is preferred that the apparatus includes a controller and at least one sensor for measuring a physical property related to the compost heap, for example the moisture content, temperature or other property within the compost heap or in the air drawn from the compost heap. The controller receiving a signal from the at least one sensor, comparing that signal with a desired signal and controlling the air moving means to adjust the flow rate of air through the aeration holes in response to said signal. The controller preferably receives signals from a plurality of sensors.

The ventilation pipes may be of any suitable size, but are preferably substantially straight and have a diameter between 60 mm and 100 mm. The diameter depends upon the depth of the heap, with a deeper heap preferably being provided with a wider ventilation pipe. The ventilation portion may comprise the entire length of the pipe within the heap, but preferably is between 500 cm and 1500 cm in length and includes a plurality of ventilation apertures of between 6 mm and 25 mm in diameter. The length of the ventilation portion is preferably about 500 cm for a 3 m heap, with the length increasing to 1500 cm for a 5 m heap. The total area of ventilation apertures is preferably selected so that it is not greater than the cross sectional area of the ventilation pipe. There is preferably one ventilation pipe for a heap volume of between 30 m³ and 80 m³ and most preferably one per 50 m³.

The ventilation pipe may be made from a plastics material as such materials are relatively cheap and easy to work with and are resistant to corrosion, but the ventilation pipes may be fabricated from any suitable material and is preferably fabricated from steel as this is resistant to the forces involved in the pile driving operation.

The flow rate of air through the ventilation pipes is preferably chosen to maintain the correct aeration of the compost. This flow rate depends upon the state of composting in the heap with a newer heap requiring a higher flow rate of air which gradually reduces as the composting process proceeds. The flow of air may be controlled based upon the temperature of the heap detected by in heap temperature sensors with a higher temperature resulting in a higher flow of air. The air flow may be pulsed, with periods of constant flow separated with periods of no flow. For example, a hot heap may have 15 minutes of air flow per hour, while a cooler heap requires only 6 minutes per hour of air flow at the same rate,

### BRIEF DESCRIPTION OF THE DRAWINGS

The invention will now be further described, by way of example, with reference to the accompanying drawings, in which:

FIG. 1 shows a cross section through a windrow compost heap;

FIGS. 2a, 2b and 2c shows a cross section through a compost heap as a ventilation pipe is inserted into the compost heap;

FIG. 3 shows a cross section through an end of a ventilation pipe.

### DETAILED DESCRIPTION

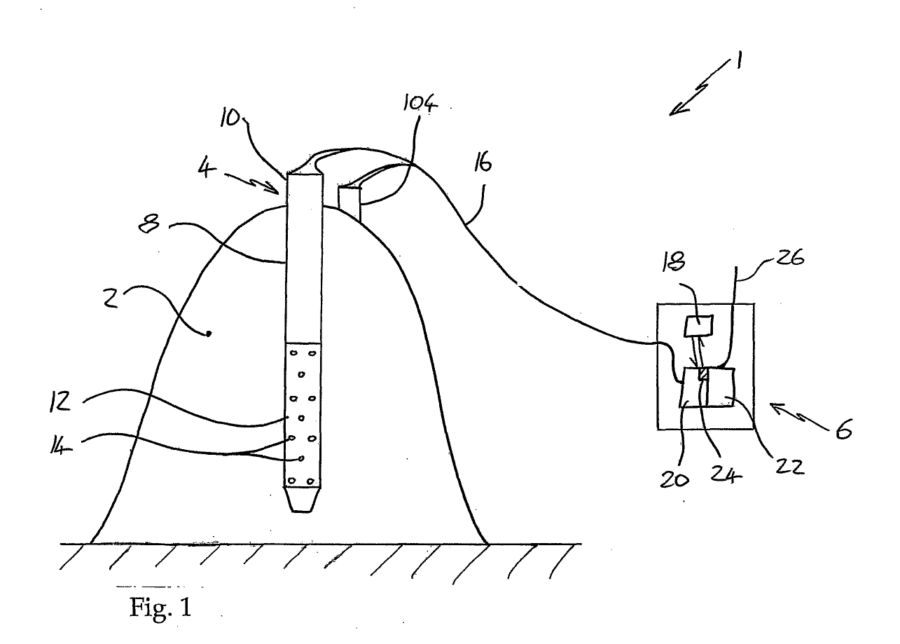

FIG. 1 shows a composting apparatus 1 comprising a compost heap 2 comprising compostable material. There is at least one ventilation pipe 4 and air moving means 6, in this case a fan. The ventilation pipe 4 comprised a pipe wall 8 defining a conduit 10 for gas. The ventilation pipe 4 has a connection end 10 and a ventilation portion 12. The ventilation portion 12 has a plurality of aeration holes 14 extending through the pipe wall 8. The ventilation portion 12 is located within the compost heap 2 and the connection end 10 is coupled to the air moving means 6 by a pipe 16 such that, when active, the air moving means 6 causes movement of air through the aeration holes 14 towards the air moving means 6. In this case the ventilation pipe is a 76 mm outer diameter steel pipe having a wall thickness of 3 mm.

The air moving means 6 comprise a controller 18, an air mover 20, a filter 22 and a sensor 24. The controller 18 receives a signal from the sensor 24 and controls the air mover 20, in this case a fan blower, in response to said signal. It should be understood that the apparatus could function without the sensor with the controller 18 simply controlling the air mover 20 at a constant speed depending upon whether the air moving means is active or not, for example the controller could be a simple switch.

The air drawn from the heap 2 is passed through filter 22 for treatment to substantially reduce the odour of the gas before it is vented to atmosphere through vent 26.

This compost heap 30 could be located inside a building on a concrete base, but could also be a windrow compost heap located outdoors on any surface, for example a surface of a field.

FIG. 1 also shows a further ventilation pipe 104 located in the compost heap 2 and coupled to the same air moving means. It is possible that each ventilation pipe could have its own air moving means, but a single air moving means is preferred.

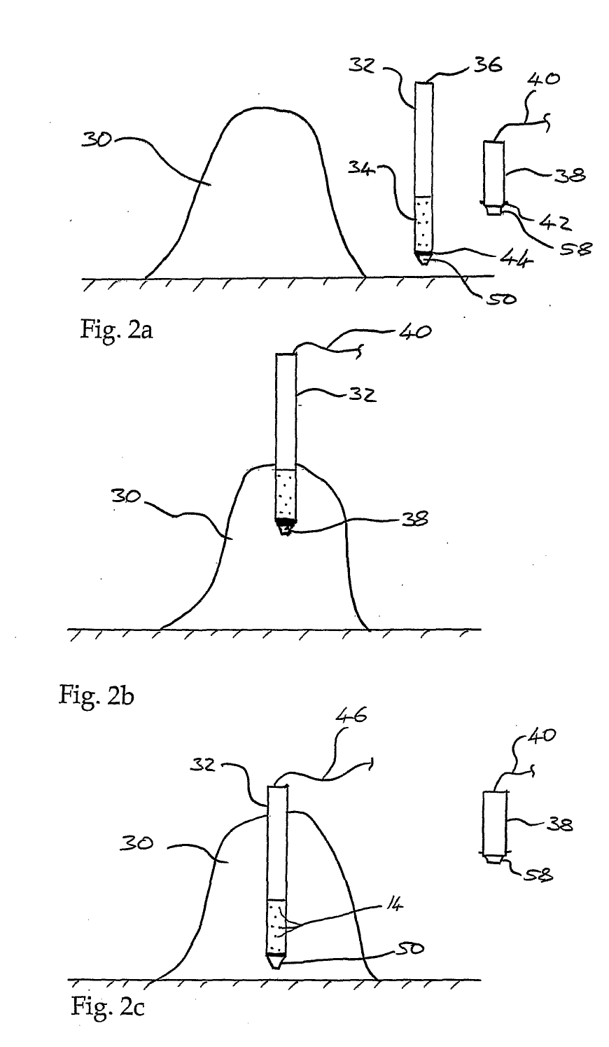

FIG. 2a shows an initial stage in the ventilation of a compost heap 30 using a ventilation pipe 32 having a ventilation portion 34 and a connection end 36. A compressed air powered piler 38 dimensioned so as to fit through the ventilation pipe 32 is provided to create the hole in which the ventilation pipe 32 will be arranged. The piler 38 includes an air line 40 leading to a source of compressed air (not shown).

The piler 38 includes a coupling means 42 which includes a taper 58 adapted to couple to coupling means 44 in the nose 50 at an end of the ventilation pipe opposite the connection end 36. the coupling means 44 include a female taper 56 which is adapted to engage with the taper 58.

FIG. 2b shows the ventilation pipe 32 and piler 38 coupled together by engagement between the coupling means 42 and 44. The piler 38 is acting as an internal pile driver within the ventilation pipe 4 and driving it into the compost heap 30. This process will continue until the ventilation pipe 32 has reached a desired depth.

FIG. 2c shows the ventilation pipe 32 in position within the compost heap 30. The coupling means 42,44 have been disengaged and the piler 38 retrieved from the ventilation pipe 32. A compressed air line 46 is coupled to the connection end 36 and compressed air blown into the ventilation pipe 32 to blow debris from the aeration holes 14. The air line 46 is then removed and the connection end 36 connected to air moving means as shown in FIG. 1.

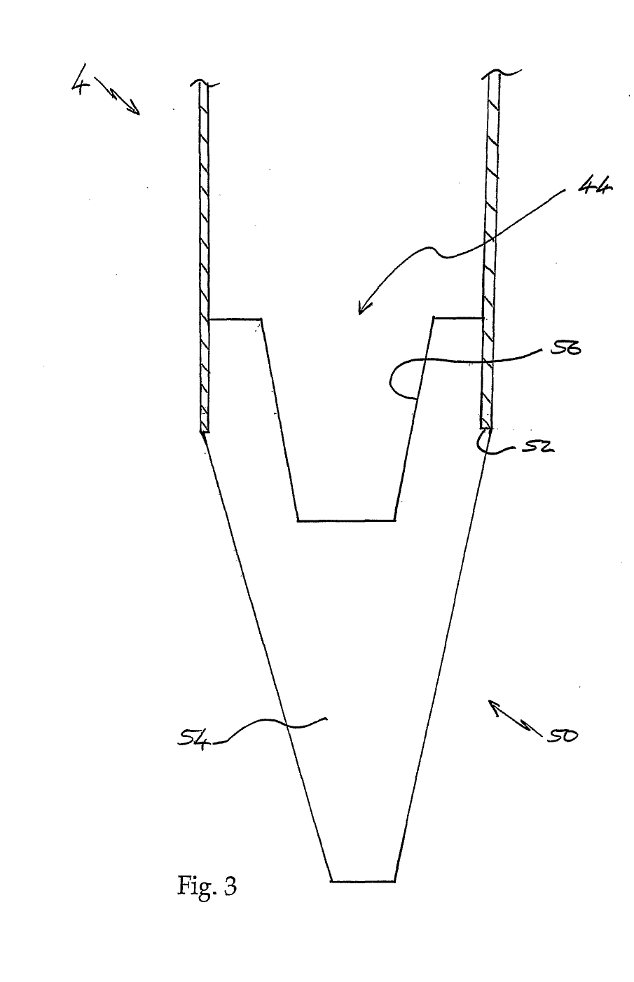

FIG. 3 shows a ventilation pipe 4 having a nose portion 50 adapted to penetrate the compost heap during the piling operation. The nose portion 50 includes a shoulder 52 which contacts the wall 8 of the ventilation pipe 4 and is welded thereto to secure the nose portion 50 to the ventilation pipe 4. The nose portion comprises a substantially solid machined truncated cone shaped tip 54 which allows for increased penetration of the ventilation pipe 4 into the compost heap 2 during the piling operation. The nose portion further includes a male taper section 56 which engages with a female protrusion of the piler apparatus 58 to form a Morse taper type engagement although other engagement means could be used. This outer shape of the tip 54 is one example of an adaptation to enable deeper penetration into the compost. The tip 54 preferably has a tapered shape such that a leading end has a smaller cross sectional area than the ventilation pipe. The leading end could be pointed or truncated and the cross section could be any suitable shape such as star, square, oval or circular. The nose portion may be integrally formed with the ventilation pipe, or attached by welding, or attachment means such as a screw thread or bayonet fitting. The nose portion is preferably substantially solid.

It should be understood that the invention has been described above by way of example only and that modifications in detail may be made without departing from the scope of the invention as described in the claims.

1. A composting apparatus comprising a compost heap comprising compostable material, at least one ventilation pipe and air moving means, the ventilation pipe comprising a pipe wall defining a conduit for gas and the ventilation pipe having a connection end and a ventilation portion, the ventilation portion having a plurality of aeration holes extending through the pipe wall and wherein the ventilation portion is located within the compost heap and the connection end is coupled to the air moving means such that, when active, the air moving means draws air from the, or each, ventilation pipe; and the apparatus further comprising air treatment means for treating the air drawn from the ventilation pipe to reduce odours in the gas before it is released to atmosphere.

2. A composting apparatus according to claim 1, in which the compost heap has an external surface, through which air is drawn to the ventilation pipe.

3. A composting apparatus according to claim 1, in which the ventilation portion is positioned below the top core such that the air is drawn through the top core to the ventilation pipe.

4. A composting apparatus according to claim 1, in which there is a plurality of ventilation pipes, each ventilation pipe having a ventilation portion located within the compost heap and a connection end connected to the air moving means such that, when active, the air moving means causes movement of air through the aeration holes of each ventilation pipe.

5-9. (canceled)

10. A composting apparatus according to claim 1, in which the at least one ventilation pipe is arranged such that it extends substantially vertically with the connection end extending above the compost heap.

11. A composting apparatus according to claim 1, in which the ventilation portion is at or adjacent an end of the, or each, ventilation pipe opposite the connection end.

12. A method of ventilating a compost heap using at least one ventilation pipe and an air moving means, the ventilation pipe comprising a pipe wall defining a conduit for gas and the ventilation pipe having a connection end and a ventilation portion, the ventilation portion having a plurality of aeration holes extending through the pipe wall and the method comprising the steps of:
constructing a compost heap comprising compostable materials;
inserting the ventilation portion of the at least one ventilation pipe into the compost heap;
connecting the connection end of the ventilation pipe to the air moving means;
using the air moving means to draw air from the ventilation pipe and draw air through the aeration holes into the ventilation pipe via towards air treatment means.

13. A method according to claim 12, in which the constructing step of comprises constructing a compost heap having an external surface, and step of using the air moving means to draw air from the ventilation pipe comprises drawing air through external surface towards the aeration holes and into the ventilation pipe

14. A method according to claim 12, in which the step of inserting the ventilation portion of the at least one ventilation pipe comprises inserting the ventilation portion below the top core of the compost heap.

15. A method according to claim 12, in which the step of inserting the ventilation portion into the compost heap comprises the steps of:
coupling ventilation pipe to a piler apparatus;
using the piler apparatus to drive the ventilation pipe into the compost heap;
releasing the piler apparatus from the ventilation pipe; and
retrieving the piler apparatus from the ventilation pipe.

16. A method according to claim 15, in which the piler apparatus is powered by compressed air.

17. A method according to claim 12, in which compressed air is blown into the ventilation pipe after insertion such that air is forced out through the aeration holes substantially clearing them of debris that may hinder air flow.
